(19) United States
(12) Patent Application Publication
(10) Pub. No.: US 2019/0099234 A1
(43) Pub. Date: Apr. 4, 2019

(54) PACKAGED ARTICLE

(71) Applicant: DEPUY IRELAND UNLIMITED COMPANY, COUNTY CORK (IE)

(72) Inventors: DUNCAN G. YOUNG, MELBOURN (GB); PATRICK CANNON, WARSAW, IN (US); SUSAN DIAMOND, FORT WAYNE, IN (US)

(21) Appl. No.: 15/723,449

(22) Filed: Oct. 3, 2017

(51) Int. Cl. A61B 50/30; A61F 2/00; B65D 77/20

(52) U.S. Cl. CPC A61B 50/30; B65D 77/2024; A61F 2/0095

(57) ABSTRACT

The packaged article includes first and second members that define a sealed interior cavity in which the article is housed. The packaged article further includes a continuous seal that loins a portion of the first member and the second member, the seal having spaced apart first and second ends. The first and second members can be separated by peeling one of the members from the other member in a direction along the length of the seal from the first end towards the second end. The force required to separate the members at each of first, third and fifth regions of the seal which are spaced apart along the length of the seal between the first and second ends of the seal is different from the force required to separate the members at each of second and fourth regions which are located between the first and third regions and third and fifth regions respectively.

### TECHNICAL FIELD OF THE INVENTION

The present invention relates to a packaged article in which the package includes first and second members which can be separated from one another to allow access to the article located within the package.

### BACKGROUND

A user is expected to maintain an aseptic transfer technique when opening a sealed package which contains a sterile medical component during a surgical procedure. The force required to overcome the seal strength can make it difficult to control the step of opening the package, with the possibility that the component falling out of the package. This could lead to the sterility of the component being compromised. In addition, the article might be damaged physically by uncontrolled impact.

### SUMMARY OF THE INVENTION

The invention provides a packaged article in which the force that is required to separate first and second members of the package varies along the length of the package.

The invention therefore provides a packaged article, comprising:

first and second members that define a sealed interior cavity in which the article is housed, and

a continuous seal that joins a portion of the first member and the second member, the seal having spaced apart first and second ends,

in which:

the first and second members can be separated by peeling one of the members from the other member in a direction along the length of the seal from the first end towards the second end,

the force required to separate the members at each of first, third and fifth regions of the seal which are spaced apart along the length of the seal between the first and second ends of the seal is different from the force required to separate the members at each of second and fourth regions which are located between the first and third regions and third and fifth regions respectively, and in which the ratios of each of the distances between the centres of the portions of the seal in the first and third regions at which the separation force is at a maximum and between the centres of the portions of the seal in the third and fifth regions at which the separation force is at a maximum, to the distance between the first and second ends of the seal, is at least about 0.1.

The package provided by the first and second members has the advantage that a user is provided with indications as to the extent to which the separation of the members has progressed. The indications are provided in the form of changes to the resistance to separation as the members are progressively separated. This can help the user to apply separating force to the package to separate the first and second members in a more controlled way compared with packages in which the resistance to separation is constant. The distance between the high resistance first and third regions, and between the high resistance third and fifth regions of the seal, relative to the length of the seal, means that a user is readily able to identify the indications provided by the low resistance second and fourth regions.

In many constructions, the force required to separate the first and second members is greater in the first, third and fifth regions of the seal than at the second and fourth regions of the seal. The separation force can vary to a minimum value at the second region compared with the separation forces at the first and third regions. The separation force can vary to a maximum value at the third region compared with the separation forces at the second and fourth regions. The separation force can vary to a minimum value at the fourth region compared with the separation forces at the third and fifth regions. The package can include additional regions of the seal, for example with a minimum separation force at a sixth region beyond the fifth region, and a maximum separation force at a seventh region beyond the sixth region.

The force required to separate the first and second members can be less in the first, third and fifth regions of the seal than at the second and fourth regions of the seal. The separation force can vary to a maximum value at the second region compared with the separation forces at the first and third regions. The separation force can vary to a minimum value at the third region compared with the separation forces at the second and fourth regions. The separation force can vary to a minimum value at the fourth region compared with the separation forces at the third and fifth regions. The package can include additional regions of the seal, for example with a maximum separation force at a sixth region beyond the fifth region, and a minimum separation force at a seventh region beyond the sixth region.

The force that is required to separate the first and second members can be different at the first region of the seal compared with the separation force at the third region of the seal. The force that is required to separate the first and second members can be different at the third region of the seal compared with the separation force at the fifth region of the seal. The force that is required to separate the first and second members can be different at the second of the seal compared with the separation force at the fourth region of the seal. For example, the force required to separate the members at the third region of the seal can be greater than the force required to separate the members at the first region of the seal. The force required to separate the members at the fifth region of the seal can be greater than the force required to separate the members at the third region of the seal.

Optionally, the force required to separate the members at the second region of the seal is approximately equal to the force required to separate the members at the fourth region of the seal.

At least one, optionally each of two or three, of the first, third and fifth regions of the seal can be constructed so that the force required to separate the first and second members is approximately constant along a portion of the length of the seal.

The force required to separate the first and second members can be constant along one or along each of more than one of the high separation force regions (such as the first, third and fifth regions). The force required to separate the first and second members can be constant along one or along each of more than one of the low separation force regions (such as the second and fourth regions).

The force required to separate the first and second members can vary continuously along one or more portions of the length of the seal. For example, the separation force can vary continuously from a portion of the seal in a high separation force region at which the separation force is at a maximum to a portion of the seal in a low separation force region at which the separation force is at a minimum. The separation force can then vary continuously from that portion in the low separation force region to a portion of the high separation force region on the opposite side of the low separation force region. The portion of the seal at which the separation force is at a minimum can extend along a portion the seal between the portions of the seal in which the separation force varies continuously. The portion of the seal at which the separation force is at a maximum can extend along a portion the seal between the portions of the seal in which the separation force varies continuously. The separation force can vary continuously between adjacent high separation force regions of the seal so that the portion of the seal at which the separation force is at a minimum does not extend significantly along the length of the seal.

The seal will generally extend along the package provided by the first and second members from one end of the package to the other end. It will often be preferred that the seal is continuous so that the package is sealed against ingress of contaminants. The length of the seal can be measured between the ends of the package. When the seal extending along the package does not extend in a straight line between its ends, the length should be measured along the seal, rather than along a straight line extending between its ends.

Frequently, the package will have first and second seals which are arranged along opposite spaced apart edges of the package. Each of the seals can have the features specified above with multiple regions of the seal having different separation forces. A package having first and second seals along opposite spaced apart edges of the package can be opened be separating the first and second members along the two spaced apart edges substantially simultaneously.

A package can have first and second seals which are arranged along opposite spaced apart edges of the package, with one of the seals having the features discussed above where multiple regions of the seal have different separation forces, and the other of the seals being configured without the seal regions which have those differing separation forces. This can help the user to discern the individual regions of the seal where the separation forces vary.

A seal can be provided along an edge of a package which extends between opposite spaced apart edges on which the first and second seals are provided.

First and second members can be provided with tabs or other features which allow the members to be gripped to facilitate the application of a separating force.

Optionally, the ratio of the distances between the centres of the portions of the seal in the first and third regions at which the separation force is at a maximum and between the centres of the portions of the seal in the third and fifth regions at which the separation force is at a maximum, to the distance between the first and second ends of the seal is at least about 0.15, for example at least about 0.2. Optionally, the ratio of the distances between the centres of the first and third regions and between the centres of the third and fifth regions to the distance between the first and second ends of the seal is not more than about 0.5, for example not more than about 0.4.

Optionally, the force required to separate the members at each of the first, third and fifth regions of the seal is greater than the force which is required to separate the members at each of the second and fourth regions. A user will be able to discern two instances during the package opening process at which the resistance to separation of the first and second members is reduced.

Optionally, the seal has more than five regions with alternating levels of resistance to separation.

Optionally, the variation in the force required to separate the members between adjacent regions of the seal is achieved by varying the effective surface area of the seal. Accordingly, the surface area per unit length of the seal at each of the first, third and fifth regions measured between its longitudinal side edges can be greater than the surface area per unit length of the seal at each of the second and fourth regions. It is also envisaged that the surface area per unit length of the seal at each of the first, third and fifth regions measured between its longitudinal side edges might be less than the surface area per unit length of the seal at each of the second and fourth regions.

The variation in the surface area per unit length of the seal between adjacent regions of the seal can be achieved by varying the width of the seal. Accordingly, the width of the seal at each of the first, third and fifth regions measured between its longitudinal side edges can be greater than the width of the seal at each of the second and fourth regions. It is also envisaged that the width of the seal at each of the first, third and fifth regions measured between its longitudinal side edges might be less than the width of the seal at each of the second and fourth regions.

The variation in the width of the seal might be achieved by varying the width of the surface of one of the members which contacts the other member. For example when the seal surface on one of the members is provided by a flange, the width of the seal might be varied by varying the width of the flange, optionally with the seal being formed across substantially all of the width of the flange.

The variation in the width of the seal might be achieved by varying the area of the one of the members over which the seal is formed. For example, when the seal is formed by means of an adhesive, the adhesive might be coated over an area of one of the members whose width varies along the length of the seal. When the seal is formed by the application of heat or pressure or both, the area over which the heat or pressure is applied might be varied along the length of the seal, for example by means of an appropriately shaped tool.

The variation in the surface area per unit length of the seal between adjacent regions of the seal can be achieved by providing discontinuities at each of the second and fourth regions, so that the surface area per unit length of the seal is less than that at the first, third and fifth regions. The discontinuities can be provided at regions within the area of the seal in which a bond between the first and second members is not formed. For example, when the seal is provided by an adhesive, discontinuities might be provided by leaving one or more regions within the area defined by the adhesive without any adhesive. Alternatively or in addition, adhesive which has been applied to one of the members might be masked in one or more regions. Such arrangements can mean that the seal is formed between the first and second members over the area defined by the adhesive other than the region where no adhesive is provided or exposed. When the seal is formed by the application of heat or pressure or both, a discontinuity might be provided by applying the heat and pressure selectively so that heat or pressure or both are not provided in one or more regions, for example by means of an appropriately shaped tool.

Discontinuities might be provided by creating one or more hollow recesses in the surface of one of the members which is to contact a mating surface of the other member which does not have corresponding protrusions, so that the area of the member with the recesses that is available to contact the other member is effectively reduced by the area of the recesses.

Optionally, the seal includes a high separation force portion which is located between the fifth region of the seal and the second end of the seal. A high separation force portion can provide a limit to the extent to which the first and second members are separated progressively from the first end towards the second end of the seal. The separation force that would be required to separate the first and second members at the high separation force portion should be greater than the force that is required to separate the first and second members at any of the first, third and fifth regions of the seal. Optionally, the force that is required to separate the first and second members at the high separation force portion to the highest level of force that is required to separate the first and second members at the first, third and fifth regions of the seal is at least about 1.5, for example at least about 2.

The high separation force portion of the seal can be spaced apart from the second end of the seal. The location of the high separation force portion of the seal should be such that the opening that is created be separating the first and second members as far as the high separation force portion of the seal is sufficient to allow access to the article within the package.

The lengths of adjacent regions of the seal are measured with reference to the point along the length of the seal at which the force required to separate the first and second members is 50% of the difference between the maximum separation force (for example at one of the first, third and fifth regions of the seal) and the minimum separation force (for example at one of the second and fourth regions of the seal).

The ratio of the separation force in the higher force region to that in an adjacent lower force region can be at least about 1.3, for example at least about 1.5, or at least about 1.7. The distance along the seal over which the separation force changes should be kept as short as possible in many applications to facilitate detection of the change in the separation force by a user.

The length of the first region of the seal can be different from the lengths of one or both of the third and fifth regions of the seal. The length of the third region of the seal can be different form the lengths of one or both of the first and fifth regions of the seal. For example, the length of the first region of the seal can be less than the length of the third region of the seal. Alternatively or in addition, the length of the third region of the seal can be less than the length of the fifth region of the seal.

The length of the second region of the seal can be different from the length of the fourth region of the seal. Optionally, the length of the second region of the seal is approximately equal to the length of the fourth region of the seal.

Optionally, the lengths of each of the first, third and fifth regions of the seal is greater than the lengths of each of the second and fourth regions of the seal. For example, the ratio of the length of the longest of the first, third and fifth regions of the seal to the length of the shortest of the second and fourth regions can be at least about 1.5, especially at least about 2, and at least about 3 in some situations. The value of the ratio will often be not more than about 6, for example not more than about 4 for many applications. This can help a user to recognise the second and fourth regions during opening of the package.

Optionally, the ratio of the length of the third region of the seal to the length of second region is at least about 1.5. Optionally, the ratio of the length of the third region of the seal to the length of fourth region is at least about 1.5.

The force that is required to separate the first and second members immediately at the first end of the seal might be different from the force that is required to separate the members in the first of the regions of the seal. For example, the separation force might initially be high when the package has a seal along an edge which extends across the width of the package, between first and second seals which extend along the length of the package. The separation force might increase gradually from a low value, for example when the first and second members are initially separated at a corner. Portions of the seal where the first and second members are initially separated, and in which the variation in the separation force is attributable to that initial separation, are disregarded when measuring the length of the first portion of the seal.

Optionally, the first member is a base which has a recess formed in it in which the article can fit, and the second member is a cover, and in which the first and second members can be separated by peeling the cover from the base.

One or each of the first and second members can be formed by moulding. Use of a moulding technique can be appropriate when the member has a recess formed in it in which the article can be fitted. A polymeric material can be used to form a member by moulding. Use of a thermoplastic polymeric material can be convenient for many applications. Examples of suitable polymeric materials include polyolefins (such as polyethylene and polypropylene), polyesters (such as poly(ethylene terephthalates)) and polyamides.

It will often be preferred that at least one of the first and second members is made from a flexible material, especially a material which is more flexible than the material from which the other member is made. It can be preferred to use a material for a second member in the form of a cover which is more flexible than the material which is used for a first member in the form of a base. Materials which can be used for a flexible cover include films, especially made from polymeric materials, and non-woven fabrics.

The package can be used for an article which is a medical implant. For example the implant might be a component of an orthopedic joint prosthesis such as a knee joint prosthesis or a hip joint prosthesis or an ankle joint prosthesis or a shoulder joint prosthesis. The implant might be used for other applications, including for example in cardiovascular applications. Examples of other implants include guidewires, stents, pacemakers, shunts, sutures.

The seal between the first and second members can be formed without use of a material other than the materials of the first and second members. For example, the seal might be formed by the application to the members of heat or pressure or both. The application of heat can be used to cause the material of one or each of the first and second members to soften for the purpose of forming a bond. The material of one or each of the first and second member might be softened in other ways, for example by use of an appropriate solvent.

The seal between the first and second members can be formed by using a material other than the materials of the first and second members, especially an adhesive. The selection of a suitable adhesive material will depend on the materials of the first and second members. Suitable adhesive materials are known. It can be appropriate for some applications to use a pressure sensitive adhesive, such as an acrylic pressure sensitive adhesive.

Many medical implants are supplied after being sterilised. The packaging must maintain the sterility of the article until use. The materials which are used for the package must be capable of maintaining the sterility of the article.

The package can be used for an article which is an instrument or other component for used during a surgical procedure. For example, when the surgical procedure is an orthopedic surgical procedure, the instrument might be one which is used in a step to prepare a patient's bone, for example by cutting the bone or shaping the bone.

Packaging for an article should also be capable of protecting the article against damage prior to use, for example as a result of impact. A package might be provided with an appropriately shaped recess which helps to retain an article in the package in a position in which it is appropriately protected.

Appropriate materials for use in a package for a medical implant are well known, including those which can withstand the conditions to which a package is exposed during sterilisation of an article located within the package.

The seal between the first and second members should be capable of being broken to allow access to the article within the package. Generally, the seal should be capable of being broken without causing damage to either of the first and second members. The seal will generally be one which allows the package to be opened by peeling one of the members from the other member. Peeling one member from the other can involve cohesive failure of the seal or adhesive failure of the seal

It will frequently be desirable for the seal between the first and second members to extend continuously along the members so that the seal is secure against passage of fluids and other contaminants into and out of the package. This can be desirable when, for example, the article in the package has been made sterile, for example for use in a medical application such as surgery.

### BRIEF DESCRIPTION OF FIGURES

Embodiments of the invention are described below by way of example with reference to the accompanying drawings, in which:

FIG. 1a is an isometric view of a package made up of a base member and a lid member which can be used to package an article.

FIG. 1b is a graph showing how the force required to separate the base and lid members of the package differs along the length of the package;

FIG. 2a is a plan view of a second construction of a base member for a package.

FIG. 2b is a graph showing how the force required to separate a lid member from the base member differs along the length of a package constructed using this base member;

FIG. 3a is a plan view of a third construction of a base member for a package.

FIG. 3b is a graph showing how the force required to separate a lid member from the base member differs along the length of a package constructed using this base member;

FIG. 4a is a plan view of a fourth construction of a base member for a package.

FIG. 4b is a graph showing how the force required to separate a lid member from the base member differs along the length of a package constructed using this base member;

FIG. 5a is a plan view of a fifth construction of a base member for a package.

FIG. 5b is a graph showing how the force required to separate a lid member from the base member differs along the length of a package constructed using this base member;

FIG. 6a is a plan view of a sixth construction of a base member for a package.

FIG. 6b is a graph showing how the force required to separate a lid member from the base member differs along the length of a package constructed using this base member;

FIG. 7a is a plan view of a seventh construction of a base member for a package.

FIG. 7b is a graph showing how the force required to separate a lid member from the base member differs along the length of a package constructed using this base member;

### DETAILED DESCRIPTION

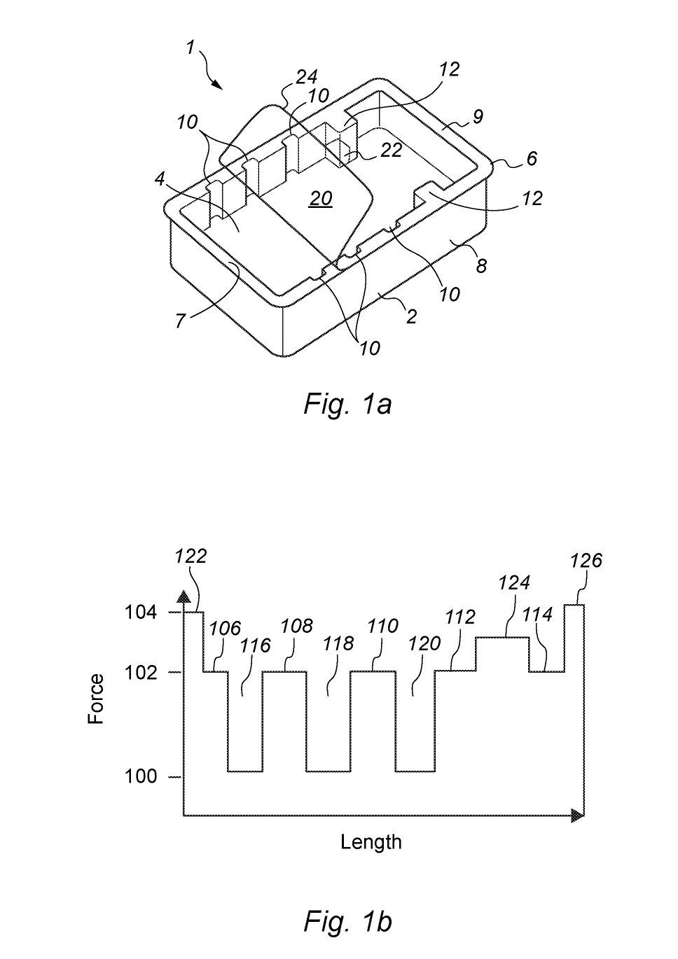

Referring to the drawings, FIG. 1a shows a package 1 which can be used for an article, to protect the article prior to use. The package 1 can also help to preserve the sterility of an article. The package 1 can be suitable for an article which is for a medical application, including for example an implant or an instrument.

The package 1 shown in FIG. 1a has a moulded base member 2. The base member 2 defines a recess 4 for an article. The base member 2 is formed from a material which is resistant to deformation so that the recess in the base member is not readily collapsed when exposed to a force. An example of a material from which the base member 2 might be made is an ethylene terephthalate polymer.

The base member 2 has a plurality of side walls 8; the side walls 8 define the sides of the recess 4. A flange 6 is located at the top of the side walls 8, extending continuously around the base member in a single plane.

Each of the side walls 8 of the base member 2 has a plurality of recesses 10 formed in it, spaced apart along the length of the base member. Each of the side walls 8 also has a buttress 12 formed in it. The width of the flange 6 varies along the side walls 8 of the base member 2, being widest at the buttresses 12 and narrowest at each of the recesses 10.

The package 1 includes a lid member 20 which is formed from a flexible non-woven fabric. An example of a suitable material for the lid 20 is a non-woven fabric made from polyolefin fibres. An example of such a fabric is that sold under the trade mark TYVEK®.

The lid member 20 is fastened to the base member 2 by means of a layer of an adhesive. The adhesive is applied to the flange 6 across the full width of the flange. For many applications, the adhesive is applied to the flange 6 continuously around the base member 2 so that it can provide a continuous seal. The adhesive should be one which is able to withstand the conditions to which the package 1 is exposed between packaging an article and opening the package 1 so that the article can be used. The adhesive should be one which allows the lid member 20 to be separated from the base member 2 by peeling the lid member back. The lid member 20 has a tab 22 at a first end 24 which enables the lid member to be gripped for application of a peeling force. Examples of suitable adhesives for this application are known, and include polymers that can be blended to form peelable seal layers. Example polymers that can be blended in this manner include DuPont™Elvax® EVA which is blended with polybutene, and also DuPont™Surlyn® which can be blended with DuPont™Bynel®.

The package has a seal which extends along the width of the package between the regions of the seal which extend along the length of the package. The package is opened initially along the edge 7 which extends along the width of the package. The package is fully opened when the seal along the edge 9, which extends along the width of the package, is broken.

The force required to separate the lid member 20 from the base member 2 varies along the length of the base member, as shown in FIG. 1b. The separation force that is required depends on the width of the flange 6, being greatest where the flange is widest and least where the flange is narrowest. Accordingly, the force that is required to separate the lid member 20 from the base member 2 is less at the points where the lid member is being separated from the portions of the flange 6 which coincide with the recesses 10 compared with the force that is required to separate the lid member from the portions of the flange on each side of the recesses. The force that is required to separate the lid member 20 from the base member 2 is greater at the buttresses 12 so that it is difficult to separate the lid member from the base member beyond the buttress.

In use, the lid member 20 is gripped by a user using the tab 22 at a first end 24 and a peeling force is applied to the lid member, so that the lid member is gradually peeled back from the base member 2. Once the user has separated the lid member 20 from the base member 2 at the first end 24 of the lid member, the force required to separate the lid and base members is approximately constant as the lid member is peeled back towards the buttresses 12, apart from three small reductions in the separating force when the lid and base member are separated at the positions corresponding to the recesses 10. The reductions in the separating force can help the user to monitor the progress of the package opening step, and therefore to gauge the force that is being applied to the package 1 to open it.

The separation force levels are illustrated schematically in FIG. 1b. As shown in FIG. 1b the package 1 has a separation force that varies between a low level separation force 100, a moderate level separation force 102 and a high level separation force 104.

The force which is required to separate the lid member 20 from the base member 2 along the first edge 7 is high (reference numeral 122 in FIG. 1b). This is because the user has to break the seal across the whole of the first edge 7 when opening the lid using the tab 22. The separation force is at a moderate level in five regions 106, 108, 110, 112, 114 along the length of the package. The moderate separation force regions 106, 108, 110, 112 are separated by three low separation force regions 116, 118, 120. These low level separation force regions coincide with the recesses 10. The moderate separation force regions 112, 114 are separated by a separation force region 124. This separation force region 124 coincides with the buttresses 12. This region has a higher separation force than the moderate separation force regions 106, 108, 110, 112, 114, but the separation force in this region is lower than the separation force required to peel the seal at the edges 7 and 9 (reference numeral 122, 126 respectively in FIG. 1b).

The seal between the lid and base members 20, 2 might be formed other than by means of an adhesive. For example, the seal might be formed by a welding technique, for example by the use of a solvent or by the application of heat, to soften the material of one of the contacting surfaces of the lid and base members 20, 2 followed by the application of pressure. For many applications, it will be preferred to form the seal between the lid and base members 2, 20 using an adhesive such as a pressure sensitive adhesive. In the construction shown in FIG. 1, the seal is preferably formed over the entire width of the flange 6, all around the base member 2.

The recesses 10 might be provided along just one of the sides of the package 1 instead of being provided along both sides.

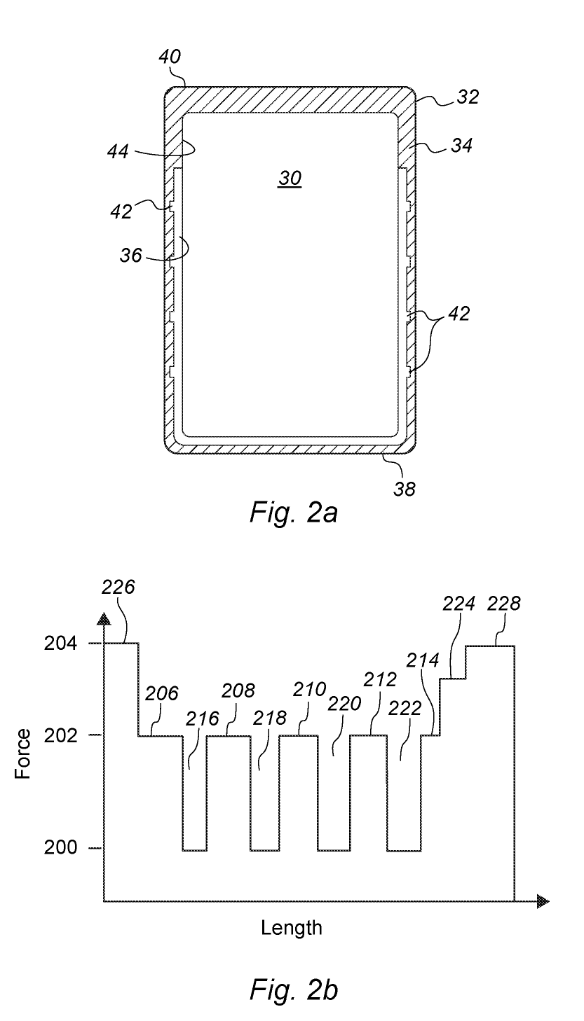

FIG. 2 is a plan view of a base member 30 of the general kind shown in FIG. 1. The base member has a flange 32 whose width is constant around the base member. In the drawing, an outer portion 34 of the flange 32 is shaded, contrasting with an inner portion 36. The outer portion 34 of the flange 32 is the portion to which a seal is made to a lid member (shown as 20 in FIG. 1), for example by selective application of a pressure sensitive adhesive, or by selective softening of the material of the base member with a view to forming a weld.

It can be seen that the width of the shaded outer portion 34 is less at a first end 38 of the base member 30 than at the opposite second end 40. The width of the shaded outer portion 34 of the flange 32 along the sides of the base member 30 is approximately equal to that along the first end 38, along most of the lengths of the sides except for four points at which there are notches 42 in the shaded outer portion 34, and the ends 44 of the sides adjacent to the second end 40 of the base member where the width of the shaded outer portion is the same as that along the second end 40.

The selection of the area of the flange 42 over which a seal is formed between the base member and a lid member has a similar effect as the shaping of the flange which is shown in FIG. 1b.

In use, a lid member (shown as 20 in FIG. 1) is gripped by a user using a tab (shown as 22 in FIG. 1) and a peeling force is applied to the lid member, so that the lid member is gradually peeled back from the base member 30, starting from the first end 38 of the base member 30 towards the opposite second end 40. Once the user has separated the lid member (shown as 20 in FIG. 1) from the base member 30 at the first end 38 of the base member 30, the force required to separate the lid and base members is approximately constant as the lid member is peeled back towards the ends 44 of the sides, apart from four small reductions in the separating force when the lid and base member are separated at the positions corresponding to the notches 42.

The graph of FIG. 2b is a schematic of the qualitative force separation levels felt by a user of a package that includes a base member 30 as shown in FIG. 2a when a lid member is peeled away from the base member. The force separation levels vary between a low level separation force 200, a moderate level separation force 202 and a high level separation force 204.

The separation force which is required to peel the lid away from the base member 30 along the first end is high (reference numeral 226 in FIG. 2b). The separation force is at a moderate level in five regions 206, 208, 210, 212, 214 along the length of the package. The moderate level separation force regions 206, 208, 210, 212, 214 are separated by four low separation force regions 216, 218, 220, 222. These low level separation force regions coincide with the notches 42. A higher level separation force region 224 coincides with the ends 44 of the sides adjacent to the second end 40 of the base member where the width of the shaded outer portion is the same as that along the second end 40. When the user peels the lid at the edge 40, sh/e will feel an increase in the force separation level (reference numeral 228 in FIG. 2b) to the same high force separation level as was felt when peeling the lid from the base at the edge 38. However, this high force separation level 228 will last for longer than the high force separation level 226, as the length of the seal at the second end 40 is longer than the length of the seal at the first end 38.

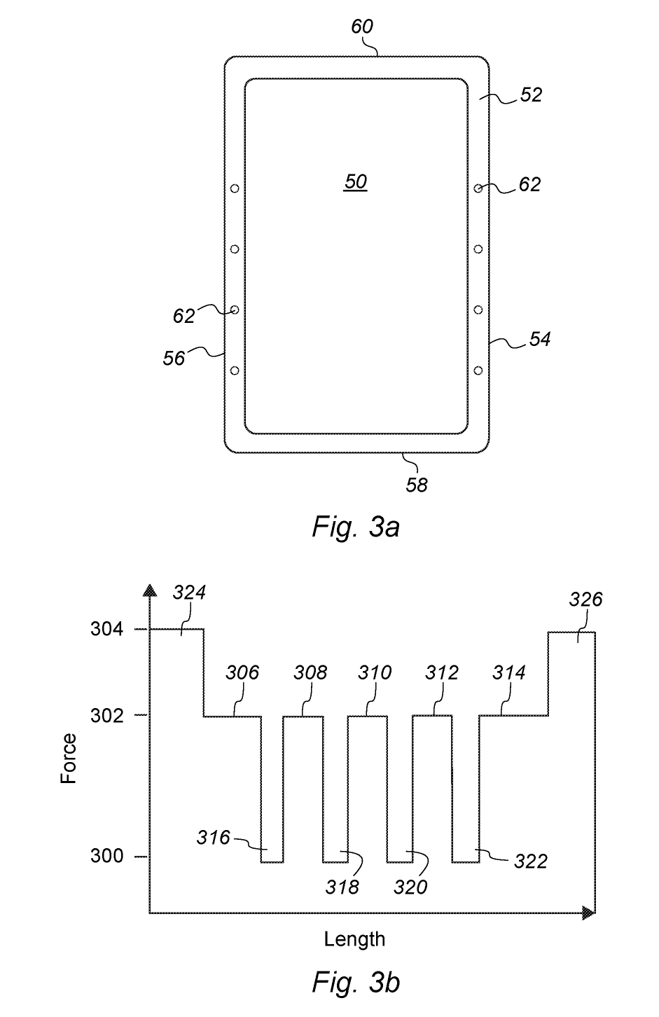

FIG. 3a is a plan view of a base member 50 of the general kind shown in FIG. 1. The base member 50 has a flange 52 having a constant width. The flange has side walls 54, 56, a first end 58 and an opposite second end 60.

A coating of a pressure sensitive adhesive is provided over the entire area of the flange 52 with the exception of four spaced apart small circular patches 62 on each of the long sides of the flange. The adhesive can be omitted from the small patches 62 in any of a number of ways including, for example, by masking those patches of the flange 52 when spraying an adhesive on to the flange, or by applying an appropriately shaped sheet of an adhesive material to the flange where the patches have been cut out of the sheet. The flange 52 might also by shaped with depressions in the flange at the locations of the circular patches 62 so that a lid member which is applied to the flange does not contact the flange at the locations of the patches. A user of a package, which incorporates the base member 50, shown in FIG. 3a would detect four small reductions in the levels of separating force when the lid and base members are separated at the positions corresponding to the circular patches 62.

In use, a lid member (shown as 20 in FIG. 1) is gripped by a user using a tab (shown as 22 in FIG. 1) and a peeling force is applied to the lid member, so that the lid member is gradually peeled back from the base member 50, starting from the first end 58 of the base member 50 towards the opposite second end 60. Once the user has separated the lid member from the base member 50 at the first end 58 of the base member 50, the force required to separate the lid and base members is approximately constant as the lid member is peeled back towards the second end 60, apart from four small reductions in the separating force when the lid and base member are separated at the positions corresponding to the circular patches 62.

The graph of FIG. 3b is a schematic of the qualitative force separation levels felt by a user when a lid member is peeled away from the base member 50 shown in FIG. 3a. The force separation levels vary between a low level separation force 300, a moderate level separation force 302 and a high level separation force 304.

The separation force which is required to peel a lid from the base member 50 along the first end 58 is high (reference numeral 324 in FIG. 3b). The separation force is at a moderate level in five regions 306, 308, 310, 312, 314 along the length of the package. The moderate level separation force regions 306, 308, 310, 312, 314 are separated by four low separation force regions 316, 318, 320, 322. These low level separation force regions coincide with the small circular patches 54 on each of the long sides of the flange.

When the user peels the lid at the edge 60, sh/e will feel an increase in the force separation level to the same high force separation level as was felt when peeling the lid from the base at the edge 58 (reference numeral 326 in FIG. 3b). This high force separation level 326 will last for the same length of peel as the high force separation level 224, as the length of the seal at the second end 60 of the base member 50 is the same length as the length of the seal at the first end 58 of the base member.

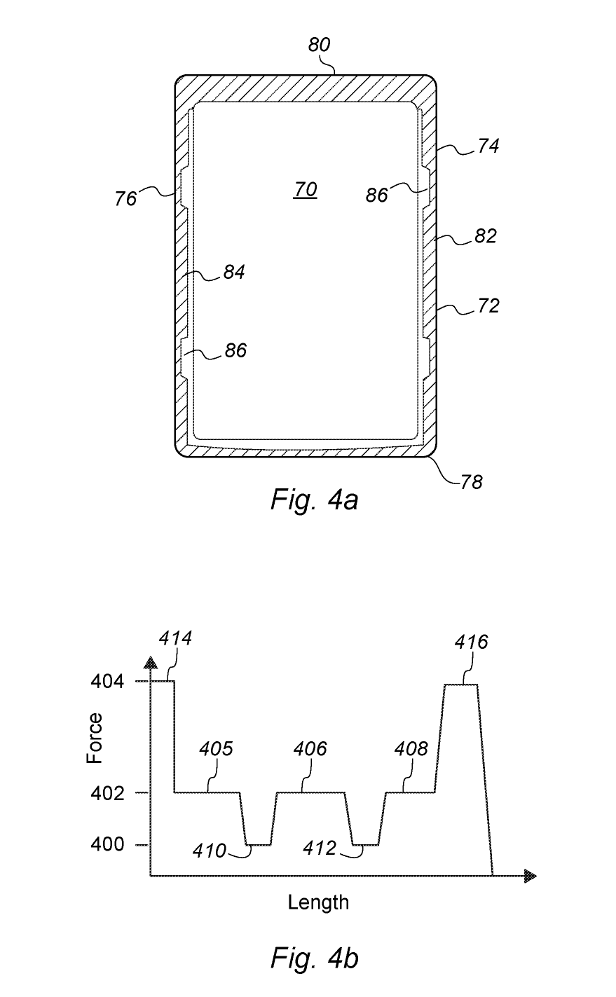

FIG. 4 is a plan view of a fourth construction of a base member 70 of the general kind shown in FIG. 1. The base member 70 has a flange 72 having a constant width. The flange has side walls 74, 76, a first end 78 and an opposite second end 80.

In the drawing, an outer portion 82 of the flange 72 is shaded, contrasting with an inner portion 84. The outer portion 82 of the flange 72 is the portion to which a seal is made to a lid member (shown as 20 in FIG. 1), for example by selective application of a pressure sensitive adhesive, or by selective softening of the material of the base member with a view to forming a weld.

It can be seen that the width of the shaded outer portion 82 is less at a first end 78 of the base member 70 than at the opposite second end 80. The width of the shaded outer portion 82 of the flange 72 along the sides of the base member 70 is approximately equal to that along the first end 78, along most of the lengths of the sides 74, 76 except for two points at which there are notches 86 in the shaded outer portion 82.

FIG. 4b is a schematic of the qualitative force separation levels felt by a user when a lid member is peeled away from the base member 70 shown in FIG. 4a. The force separation levels vary between a low level separation force 400, a moderate level separation force 402 and a high level separation force 404.

The variation in the separation force between the high separation force regions, moderate separation force regions and the low separation force regions results from variations in the width of the strip of adhesive by which the base and lid members are bonded to one another along the length of the package.

The separation force which is required to peel a lid from the base member 70 along the first end 78 is high (reference numeral 414 in FIG. 4b). The separation force is at the moderate level in three regions 405, 406, 408 along the length of the package. The region 406 is longer than the regions 405, 408. The moderate separation force regions 405, 406, 408 are separated by two low separation force regions 410, 412. The two low separation level force regions 410, 412 coincide with the notches 86 in the shaded outer portion 82.

In use, the package is opened initially from the first end 78 of the base member 70 which extends across the width of the package. The force which is required to separate the base and lid members along this seal is high (reference numeral 414 in FIG. 4b).

The package has a high separation force region 416 at the second end 80 of the flange. However, the high force separation level 416 will last for longer than the high force separation level 414, as the length of the seal at the second end 80 is longer than the length of the seal at the first end 78.

The force required to separate the base and lid members in the high separation force region 414, 416 is higher than the separation force in each of the moderate separation force regions 405, 406, 408.

The ratio of the distance between the centres of the first and third regions to the distance between the first and second ends of the seal, is about 0.33. The ratio of the distance between the centres of the third and fifth regions to the distance between the first and second ends of the seal, is about 0.29.

The ratio of the separation force in the moderate separation force regions 405, 406, 408 to that in the low separation force regions 410, 412 is about 2.5. The distance along the seal over which the separation force changes between the higher and lower values is short (as can be seen by the vertical lines in the graph of FIG. 4b).

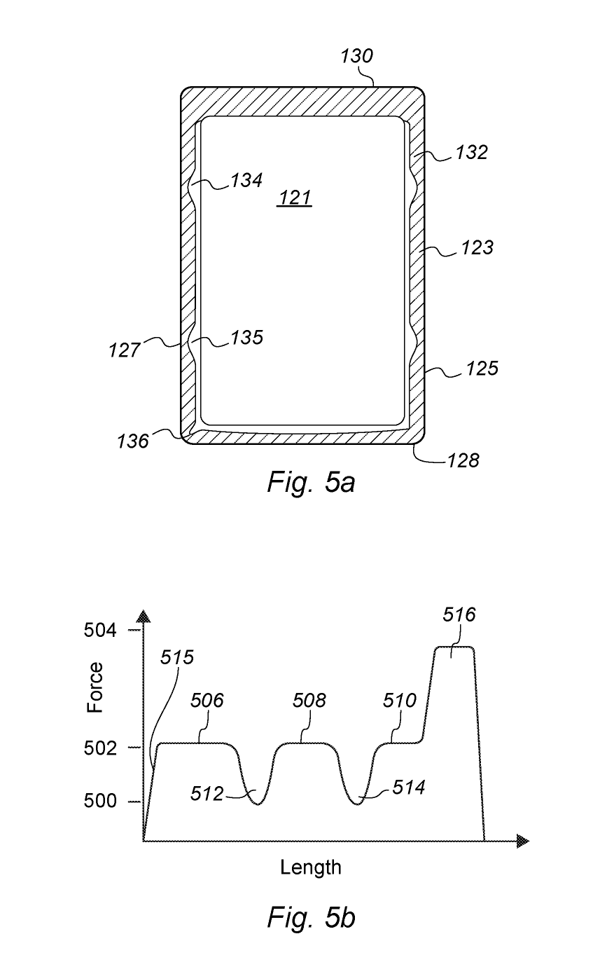

FIG. 5a is a plan view of a fifth construction of a base member 121 of the general kind shown in FIG. 1. The base member 121 has a flange 123 having a constant width. The flange has side walls 125, 127, a first end 128 and an opposite second end 130.

In the drawing, an outer portion 132 of the flange 123 is shaded, contrasting with an inner portion 135. The outer portion 132 of the flange 123 is the portion to which a seal is made to a lid member (shown as 20 in FIG. 1), for example by selective application of a pressure sensitive adhesive, or by selective softening of the material of the base member with a view to forming a weld.

It can be seen that the width of the shaded outer portion 132 is less at the first end 128 of the base member 121 than at the opposite second end 130. The width of the shaded outer portion 132 of the flange 123 along the sides of the base member 121 is approximately equal to that along the first end 128, along most of the lengths of the sides 125, 127 except for two points at which there are generally semi-circular notches 134 in the shaded outer portion 132.

The package is opened at a corner 136 where the seal area has tapered to a minimum value. Opening the package from this corner means that the force required to separate the base and lid members increases gradually from zero (reference numeral 515 in FIG. 5b). The portion of the length of the package over which the separation force increases gradually from zero is disregarded in a measurement of the length of the first region 506 of the seal along the length of the package.

FIG. 5b is a schematic of the qualitative force separation levels felt by a user when a lid member is peeled away from the base member 121 shown in FIG. 5a. The force separation levels vary between a low level separation force 500, a moderate level separation force 502 and a high level separation force 504.

The variation in the separation force between the moderate level separation force regions and the low level separation force regions results from variations in the width of the strip of adhesive by which the base and lid members are bonded to one another along the length of the package.

The separation force is at a moderate level in three regions 506, 508, 510 along the length of the package. The moderate level separation force regions 506, 508, 510 are separated by two low level separation force regions 512, 514. The two low level separation force regions 512, 514 coincide with the semi-circular notches 134 in the shaded outer portion 132.

The package has a high level separation force region 516 between the last one 510 of the moderate level separation force regions and the second end 130 of the seal. The force required to separate the first and second members in the high level separation force region is higher than the separation force in each of the moderate level separation force regions 506, 508, 510.

As can be seen from a comparison of the graphs in FIGS. 4b and 5b, the packages have a different variation in the levels of seal separation force along their length. In FIG. 4b there is an abrupt change in the levels of separation force between adjacent regions of the seal. For example, when the variation in the separation force is brought about by a change in the width of an adhesive strip, the length of the seal over which width change takes place is kept to a minimum. In contrast, as shown in FIG. 5b, the variation in the separation force between adjacent regions of the seal takes place over a greater length of the seal. This is as a result of the semi-circular shaping of the notches 134. The length of the seal over which the separation force is at a minimum is much shorter than in the FIG. 4b package. The lengths of the regions of the seal are measured between points along the length of the seal at which the separation force is 50% of the difference between the maximum separation force and the minimum separation force of two adjacent regions.

The ratio of the distance between the centres of the first and third regions to the distance between the first and second ends of the seal, is about 0.32. The ratio of the distance between the centres of the third and fifth regions to the distance between the first and second ends of the seal, is about 0.3.

The ratio of the separation force in the moderate separation force regions 506, 508, 510 to that in the low separation force regions 512, 514 is about 2.4.

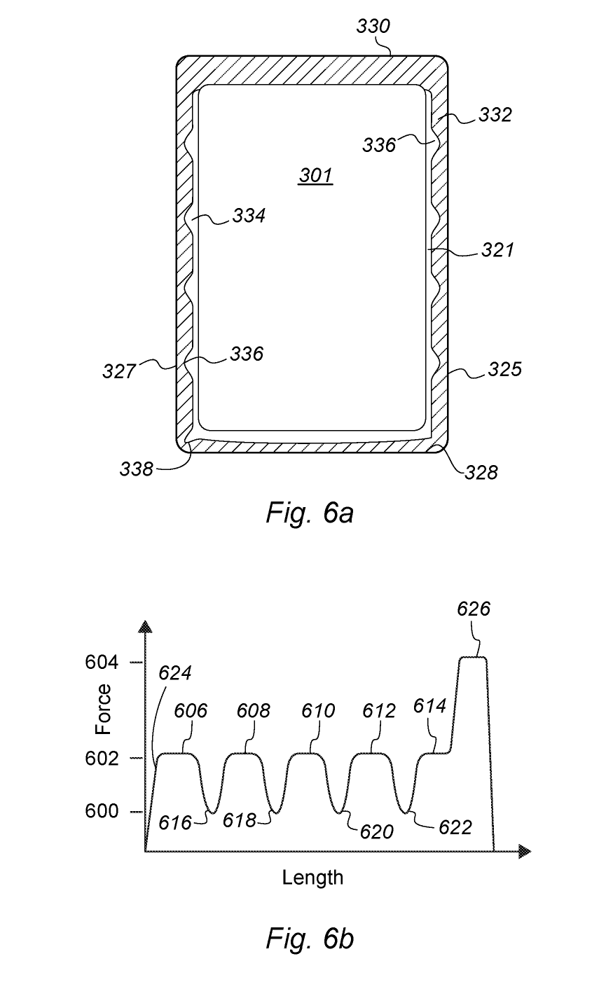

Referring to FIG. 6a, there is shown a sixth construction of a base member 301 of the general kind shown in FIG. 1. The base member 301 has a flange 321 having a constant width. The flange has side walls 325, 327, a first end 328 and an opposite second end 330.

In the drawing, an outer portion 332 of the flange 321 is shaded, contrasting with an inner portion 334. The outer portion 332 of the flange 321 is the portion to which a seal is made to a lid member (shown as 20 in FIG. 1), for example by selective application of a pressure sensitive adhesive, or by selective softening of the material of the base member with a view to forming a weld.

It can be seen that the width of the shaded outer portion 332 is less at the first end 328 of the base member 301 than at the opposite second end 330. The width of the shaded outer portion 332 of the flange 321 along the sides of the base member 301 is approximately equal to that along the first end 328, along most of the lengths of the sides 325, 327 except for four points at which there are semi-circular notches 336 in the shaded outer portion 332.

The package is opened at a corner 338 where the seal area has tapered to a minimum value. Opening the package from this corner means that the force required to separate the base and lid members increases gradually from zero (reference numeral 624 in FIG. 6b).

FIG. 6b is a schematic of the qualitative force separation levels felt by a user when a lid member is peeled away from the base member 301 shown in FIG. 6a. The force separation levels vary between a low level separation force 600, a moderate level separation force 602 and a high level separation force 604.

The variation in the separation force between the high level separation force regions, the moderate level separation force regions and the low separation force regions results from variations in the width of the strip of adhesive by which the base and lid members are bonded to one another along the length of the package.

The separation force is at the moderate level in five regions 606, 608, 610, 612, 614 along the length of the package. The moderate level separation force regions 606, 608, 610, 612, 614 are separated by four low level separation force regions 616, 618, 620, 622.

The package has a high separation force portion 626 between the last one 614 of the moderate level separation force regions and the second end of the seal 330. The force required to separate the first and second members in the high level separation force region 626 is higher than the separation force in each of the moderate level separation force regions 606, 608, 610, 612, 614.

The package is opened at the corner 338 where the seal area has tapered to a minimum value. Opening the package from the corner means that the force required to separate the base and lid members increases gradually from zero (reference numeral 624 in FIG. 6b). The portion of the length of the package over which the separation force increases from zero is disregarded in a measurement of the length of the first region 606 of the seal along the length of the package.

A package having a base member 300 shown in FIG. 6a has a different variation in the seal separation force along the length of the package compared with a package having a base member 120 shown in FIG. 5a. In the FIG. 5a, the rate of change of the separation force along the length of the package from each moderate level separation force 506, 508, 510 region to the adjacent low level separation force region 512, 514 is approximately the same as the rate of change from each low level separation force region 512, 514 to the adjacent moderate level separation force region 506, 508, 510. This can be seen in FIG. 5b where the “S-shape” of the graph from moderate level to low level separation force is approximately the mirror image of the “S-shape” of the graph from low level to moderate level separation force. In contrast, in FIG. 6b, the variation in the separation force from a moderate level separation force region 606, 608, 610, 612, 614 to an adjacent low separation force region 616, 618, 620, 622 takes place over a shorter length portion of the seal, compared with the variation in the separation force from a low level separation force region 616, 618, 620, 622 to an adjacent moderate level separation force region 606, 608, 610, 612, 614.

Similar to the base member shown in FIG. 4a, the base member shown in FIG. 6a has a seal which extends along the width of the package between the regions of the seal which extend along the length of the package.

The ratio of the distance between the centres of the first and third regions to the distance between the first and second ends of the seal, is about 0.21. The ratio of the distance between the centres of the third and fifth regions to the distance between the first and second ends of the seal, is about 0.19.

The ratio of the separation force in the moderate level separation force regions to that in the low level separation force regions is about 2.2.

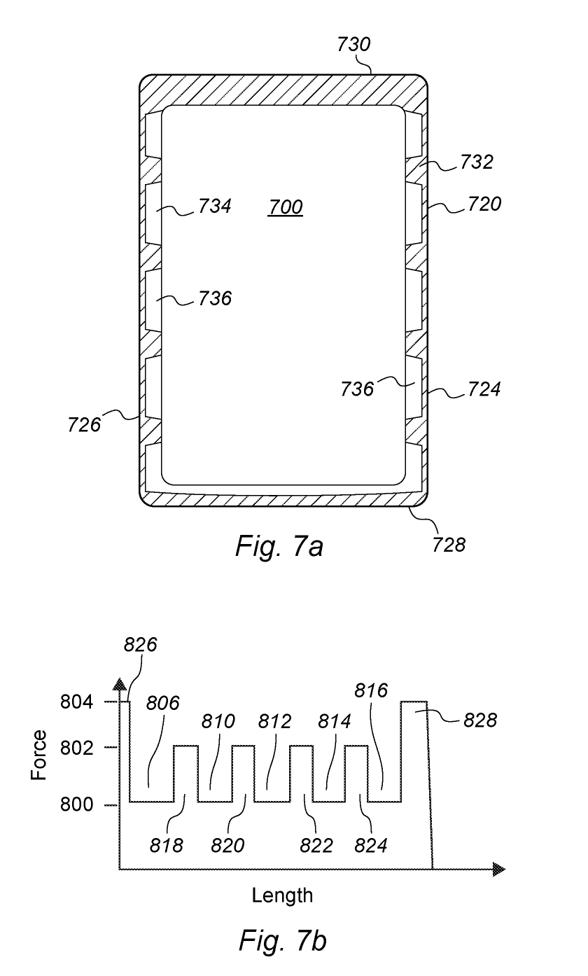

Referring to FIG. 7a, there is shown a seventh construction of a base member 700 of the general kind shown in FIG. 1. The base member 700 has a flange 720 having a constant width. The flange has side walls 724, 726, a first end 728 and an opposite second end 730.

In the drawing, an outer portion 732 of the flange 720 is shaded, contrasting with an inner portion 734. The outer portion 732 of the flange 720 is the portion to which a seal is made to a lid member (shown as 20 in FIG. 1), for example by selective application of a pressure sensitive adhesive, or by selective softening of the material of the base member with a view to forming a weld.

It can be seen that the width of the shaded outer portion 732 is less at the first end 728 of the base member 700 than at the opposite second end 730. The width of the shaded outer portion 732 of the flange 720 along the sides of the base member 700 is approximately equal to that along the first end 728, along most of the lengths of the sides 724, 726 except for four points at which there are generally rectangular recesses 736 in the shaded outer portion 732.

The force required to separate a lid member (shown as 20 in FIG. 1) from the base member 700 varies along the length of the base member, as shown in FIG. 7b. The separation force that is required depends on the width of the flange 720, being greatest where the flange is widest and least where the flange is narrowest. Accordingly, the force that is required to separate a lid member from the base member 700 is less at the points where the lid member is being separated from the portions of the flange 720 which coincide with the recesses 736 compared with the force that is required to separate the lid member from the portions of the flange on each side of the recesses.

FIG. 7b is a schematic of the qualitative force separation levels felt by a user when a lid member is peeled away from the base member 700 shown in FIG. 7a. The force separation levels vary between a low level separation force 800, a moderate level separation force 802 and a high level separation force 804.

The separation force is at a low level in five regions 806, 810, 812, 814, 816 along the length of the package. The low level separation force regions are separated by four moderate level separation force regions 818, 820, 822, 824. The variation in the separation force between the low level separation force regions and the moderate level separation force regions results from variations in the width of a strip of adhesive by which the base and lid members are bonded to one another along the length of the package.

The package has a seal which extends along the width of the package between the regions of the seal which extend along the length of the package. The package is opened initially along the edge which extends along the width of the package. The force which is required to separate the first and second members along this edge is relatively high (reference numeral 826 in FIG. 7b). This portion of the length of the package is disregarded in a measurement of the length of the first region 806 of the seal along the length of the package.

The package has a high level separation force portion 828 between the last one 816 of the low level separation force regions and the second end 730 of the seal. The force required to separate the base and lid members in the high separation force portion 828 is higher than the separation force in each of the moderate level separation force regions 818, 820, 822, 824.

The ratio of the distance between the centres of the first and third regions to the distance between the first and second ends of the seal, is about 0.20. The ratio of the distance between the centres of the third and fifth regions to the distance between the first and second ends of the seal, is about 0.19.

The ratio of the separation force in the moderate level force regions 818, 820, 822, 824 o that in the low level separation force regions 806, 810, 812, 814, 816 is about 1.9. The distance along the seal over which the separation force changes between the higher and lower values is short (as can be seen by the vertical lines in the graph of FIG. 7b).

1. A packaged article, comprising:
first and second members that define a sealed interior cavity in which the article is housed, and
a continuous seal that joins a portion of the first member and the second member, the seal having spaced apart first and second ends,
in which:
the first and second members can be separated by peeling one of the members from the other member in a direction along the length of the seal from the first end towards the second end,
the force required to separate the members at each of first, third and fifth regions of the seal which are spaced apart along the length of the seal between the first and second ends of the seal is different from the force required to separate the members at each of second and fourth regions which are located between the first and third regions and third and fifth regions respectively, and in which the ratios of each of the distances between the centres of the portions of the seal in the first and third regions at which the separation force is at a maximum and between the centres of the portions of the seal in the third and fifth regions at which the separation force is at a maximum, to the distance between the first and second ends of the seal, is at least about 0.1.

2. A packaged article as claimed in claim 1, in which the surface area per unit length of the seal at each of the first, third and fifth regions measured between its longitudinal side edges is greater than the surface area per unit length of the seal at each of the second and fourth regions.

3. A packaged article as claimed in claim 2, in which the width of the seal at each of the first, third and fifth regions measured between its longitudinal side edges is greater than the width of the seal at each of the second and fourth regions.

4. A packaged article as claimed in claim 2, in which the seal has discontinuities at each of the second and fourth regions causing the surface area per unit length of the seal to be less than that at the first, third and fifth regions.

5. A packaged article as claimed in claim 1, in which the seal includes a high separation force portion which is located between the fifth region of the seal and the second end of the seal.

6. A packaged article as claimed in claim 5, in which the high separation force portion of the seal is spaced apart from the second end of the seal.

7. A packaged article as claimed in claim 1, in which the force required to separate the members at the third region of the seal is greater than the force required to separate the members at the first region of the seal.

8. A packaged article as claimed in claim 1, in which the force required to separate the members at the second region of the seal is approximately equal to the force required to separate the members at the fourth region of the seal.

9. A packaged article as claimed in claim 1, in which the length of the first region of the seal is less than the length of the third region of the seal.

10. A packaged article as claimed in claim 1, in which the length of the second region of the seal is approximately equal to the length of the fourth region of the seal.

11. A packaged article as claimed in claim 1, in which the ratio of the length of the third region of the seal to the length of second region is at least about 1.5.

12. A packaged article as claimed in claim 1, in which the lengths of each of the first, third and fifth regions of the seal is greater than the lengths of each of the second and fourth regions of the seal.

13. A packaged article as claimed in claim 12, in which the ratio of the length of the longest of the first, third and fifth regions of the seal to the length of the shortest of the second and fourth regions is at least about 1.5

14. A packaged article as claimed in claim 1, in which the first member is a base which has a recess formed in it in which the article can fit, and the second member is a cover, and in which the first and second members can be separated by peeling the cover from the base.

15. A packaged article as claimed in claim 1, in which the article is a medical implant.
